(19) United States
(12) Patent Application Publication
(10) Pub. No.: US 2005/0022260 A1
(43) Pub. Date: Jan. 27, 2005

(54) Method of efficiently constructing transgenic birds and transgenic birds thus obtained

(76) Inventors: Shinji Iijama, Aichi (JP); Masamichi Kamihira, Aichi (JP); Kenichi Nishijima, Aichi (JP); Shinji Mizuarai, Aichi (JP); Kenichiro Ono, Aichi (JP)

Correspondence Address: Connolly Bove;Lodge & Hutz, Suite 800, 1990 M Street NW, Washington, DC, 20036-3425, US

(21) Appl. No.: 10/203,515

(22) PCT Filed: Aug. 2, 2001
(86) PCT No.: PCT/JP01/06657

(30) Foreign Application Priority Data
Dec. 12, 2000 (JP) 2000-377549

(51) Int. Cl. A01K 67/027; C12N 15/86

(52) U.S. Cl. 800/19; 435/456; 119/300

(57) ABSTRACT

The present invention relates to G₀ transgenic chimeric birds capable of transmitting a desired gene or gene arrangement to offspring with very high efficiency and to a method of producing the same, and relates to G₀ transgenic chimeric birds having a desired gene or gene arrangement in germ cells with very high efficiency and to a method of producing the same.

A G₀ transgenic chimeric bird

which comprises a G₀ transgenic chimeric bird resulting from gene transduction using a replication-defective retrovirus vector and that the efficiency of transgene transmission to G₁ is not less than 10%.

### TECHNICAL FIELD

The present invention relates to G₀ transgenic chimeric birds capable of transmitting a desired gene or gene arrangement to offspring with very high efficiency and to a method of producing the same. The present invention relates to G₀ transgenic chimeric birds having a desired gene or gene arrangement in germ cells with very high efficiency and to a method of producing the same. The present invention further relates to transgenic birds having a desired gene or gene arrangement in somatic cells and germ cells and offspring thereof and to a method of producing the same. Furthermore, the present invention provides transgenic birds having a desired gene or gene arrangement, in a greater copy number than in the prior art, in somatic cells and germ cells and offspring thereof as well as a method of producing the same. Still further, the invention provides G₀ transgenic chimeric birds, transgenic birds and offspring thereof, which are highly safe, as well as a method of producing the same. The invention further provides transgenic birds provided with an inherited character that parent birds do not have as well as a method of producing the same. Furthermore, the invention provides transgenic birds for identifying the function of a gene or the function of the protein encoded by that gene by transducing that gene, whose function is unknown, into birds as well as a method of producing the same.

### BACKGROUND ART

Transgenic animals are important in studying the function or the role in developmental stage of a gene transduced thereinto. They are also of commercial importance in providing animals with a novel character, for example in breeding of various animals or production of proteinic medicines. In particular, the idea of “animal factory” according to which a useful substance is produced in a tissue or organ of a transgenic animal is expected to provide an epoch-making method of producing the proteinic useful substance in large amounts.

Studies have so far been made on the secretory production of useful substances in the milk of transgenic animals such as goat, sheep, swine and bovine species and, actually, reports have been made on the production, in milk, of such useful substances as al-antitrypsin, blood coagulation factors or antibodies using a mammary gland-specific promoter. However, there are problems, namely large-sized mammals are slow in rate of growth and require high breeding expenditures and relatively large spaces for breeding, and for some animals, a very long period of time is required to secure a sufficient number of individuals for the commercial production of useful substances. To overcome these problems, it is desirable that transgenic birds be developed as a new production system.

The birds that are being reared as farm animals include various species, typically chickens and, further, ducks, turkeys, wild ducks, ostriches and quails and, in particular, chickens are important for the production of meat and eggs.

In the case of chickens, for instance, the technology of producing transgenic birds is applicable to breeding (e.g. growth promotion, feed efficiency, quality of eggs, meat texture, meat yield, fecundity, disease resistance, quality of feather, etc.) and production of useful substances (e.g. antigens, antibodies, physiologically active peptides, therapeutically useful proteinic medicines) in egg white, egg yolk or in other organs.

The useful substance production in avian eggs is a particularly important task in the industry. Poultry that have been improved over many years, such as chickens, grow rapidly and the number of individuals can be increased in a short period of time and, furthermore, each individual lays an egg every day, so that it will be possible to produce a useful substance continuously and in large amounts.

The egg yolk of a hen's egg has a protein content of about 20%, and the egg white has a protein content of about 10%. The main proteins in egg white, namely ovalbumin, ovotransferrin, ovomucoid and lysozyme, account for high percentages of egg white proteins, namely about 54%, 12%, 12% and 3.4%, respectively. If a useful substance can be produced as a substitute for one of such proteins, it will be possible to attain very high productivity in producing the useful substance.

However, there is no report as yet about the production of a useful substance in avian eggs. There is no report, either, about breeding of avian species which is genetically modified. The prime reason is that no method has been established for the production of G₀ transgenic chimeric birds retaining a transgene efficiently in germ cells.

Some methods have been tried for producing transgenic birds.

Thus, Love et al. (Love, J. et al. (1994), BIO/TECHNOLOGY, 12, 60) transduced a linear DNA having a marker gene into the cytoplasm of 88 eggshell-free fertile eggs collected from the oviduct of hens by microinjection and allowed them to develop and differentiate in an artificial environment. Reportedly, seven hatched out of the 88 fertile eggs, and one cock of them had the transgene in germ cells in a mosaic manner and the transgene was transmitted to 14 out of 412 chicks born of hens that had mated with the cock. However, this method requires one hen for obtaining one fertile egg, and the efficiency of transgene transmission to the next generation transgenic chimeric chickens is as low as 3.4%.

Transgenic birds were produced also by using a retrovirus vector.

Initially, the production of transgenic birds using a retrovirus vector was carried out tentatively using a replicable retrovirus vector (Salter, D. W. et al. (1987), Virology, 157, 235). When a gene is transduced into fertile eggs or embryos by means of a replicable retrovirus vector, the vector transduced infects from cell to cell and therefore it is possible to realize gene transduction without being affected by the titer of the retrovirus vector prepared. However, the pathogenicity of the replicable retrovirus vector itself to individuals cannot be denied, there is the risk of the replicable retrovirus vector transduced producing a novel pathogenic virus, and the replicable retrovirus vector transduced into individuals may possibly infect other individuals. Due to such drawbacks, it is difficult to utilize this method industrially.

Therefore, transgenic birds were produced using a replication-defective retrovirus vector.

Avian fertile eggs just after oviposition are already after ten and several times of cleavage, and the embryo is constituted of about 60,000 differentiated cells. When a replication-defective retrovirus vector is microinjected into such an embryo, the gene is transduced into a part of the cells. In the embryo at this stage (embryo at the blastoderm stage), there are cells which are to become primordial germ cells to be differentiated into germ cells in the future, and the nestling hatched from an embryo with a gene transduced into cells precursory of germ cells by microinjection has the transgene in part of the germ cells thereof (in a mosaic manner) In the present specification, such a bird is referred to as a “G₀ transgenic chimeric bird” or “G₀” for short. Further, offspring obtained from a G₀ transgenic chimeric bird by natural mating or artificial insemination (hereinafter referred to as “mating”) with a non-transgenic bird are referred to as “G₁ birds” or “G₁” for short. Among the G₁ birds, the individuals having the transgene are referred to as “G₁ transgenic birds”. Offspring obtained from G₁ transgenic birds by mating with non-transgenic birds are referred to as “G₂ birds” or “G₂” for short. Among the G₂ birds, the individuals having the transgene are referred to as “G₂ transgenic birds”. G₁ transgenic birds, G₂ transgenic birds, and those offspring obtained therefrom by mating and having the transgene are collectively referred to as “transgenic birds”. Among the offspring obtained by mating of male and female transgenic birds or transgenic chimeric birds, those individuals having the transgene are included among the transgenic birds. The offspring obtained by mating of transgenic birds undergo chromosome partitioning or gene recombination in the process of germ cell (sperm and ovum) production and thus offspring having various genotypes are born. “Offspring of ‘transgenic birds’” so referred to herein mean such transgenic birds having various genotypes.

Bosselman et al. (Bosselman, R. A. et al. (1989), Science, 243, 533) microinjected replication-defective Reticuloendotheliosis virus having the neomycin resistance gene and herpes simplex virus thymidine kinase gene into the embryo of fertile hens' eggs (a total of 2,558) just after oviposition and examined 760 out of the chicks that had hatched for the presence or absence of the transgene; 173 were positive and, in 33 males among them, the gene arrangement originating in the vector transduced was found in the sperm. The thus-selected four G₀ transgenic chimeric cocks were mated with genetically unengineered hens, and the efficiency of the transgene transmission to the thus-obtained G₁ chickens was examined and reportedly was found to be about 2% to 8%. Further, Bosselman et al. observed the formation of infectious Reticuloendotheliosis virus in two of the 14 G₀ transgenic chimeric chickens constructed by them, and this indicates that, in spite of the fact that the vector system used by them was a replication-defective retrovirus vector system, there is the risk of infectious virus formation.

Vicks et al. (Vicks, L. et al. (1993), Proc, R. Soc. Lond., B, Biol. Sci., 251, 179) isolated primordial germ cells from embryos just after oviposition, transformed them with a replication-defective retrovirus vector and transplanted them to other embryos. By this method, they obtained G₀ transgenic chimeric chickens. The efficiency of transgene transmission from G₀ they produced to G₁ is reported to be 2% or 4%, depending on the G₀ individuals obtained.

According to a review article written by Shuman, R. M. (Shuman, R. M. (1991), Experimentia, 47, 897), Lee et al. obtained 1,595 G₁ quails from one G₀ transgenic chimeric quail using a replication-defective retrovirus and the number of G₁ transgenic quails to which the transgene had been transmitted was reportedly only one.

Thoraval et al. (Thoraval, P. et al. (1995), Transgenic Res., 4, 369) obtained one G₀ transgenic chimeric chicken by transforming the chicken embryo using replication-defective avian leukosis virus having the neomycin resistance gene and Escherichia coli β-galactosidase (LacZ) gene and reported that the efficiency of transgene transmission from that chicken to G₁ was 2.7%.

As mentioned above, transgenic birds have been produced by several groups of researchers. However, the efficiency of transgene transmission to G₁ birds obtained by mating of the G₀ transgenic chimeric birds produced by their methods is low (less than 10%). It is therefore necessary to cause a large number of G₁ birds to be born and test them for the presence or absence of the transgene. This is a great obstacle to the production of transgenic birds. The low efficiency of gene transmission from G₀ transgenic chimeric birds to G₁ indicates that the number of copies of the gene transmitted to and transduced into G₁ transgenic birds is small. This is a factor restricting the productivity of useful substances in transgenic birds. As a matter of fact, there is no report available about the production of G₁ transgenic birds having a plurality of copies of a transgene using a replication-defective retrovirus vector. Even when such a replication-defective retrovirus vector as mentioned above is used, there is the risk of infectious retrovirus formation from transgenic birds and this is a great obstacle to the industrial application thereof.

Furthermore, when transgenic birds produced by using a retrovirus vector capable of infecting birds is infected with the same or a related retrovirus species, there is the risk of the provirus transduced into transgenic birds being rescued as infectious virus particles. This is a great obstacle to industrial application of transgenic birds produced by using a retrovirus vector derived from a retrovirus capable of efficiently infecting birds.

### SUMMARY OF THE INVENTION

For producing transgenic birds, it is necessary to insert a desired gene or gene arrangement into the genome of avian germ cells. The technique of DNA microinjection into the fertile egg nucleus, which is in use as a technology of producing transgenic mammals, is not in general use in producing transgenic birds because avian fertile eggs are very large, hence the position of the nucleus is difficult to locate, and because it is difficult to collect and treat fertile eggs. As a matter of fact, there is no report as yet about the production of transgenic birds by the techniques generally used in producing transgenic mammals.

For transducing a desired gene into the genome of avian germ cells, a replication-defective retrovirus vector is microinjected into avian embryos at the blastoderm stage immediately following oviposition, as mentioned hereinabove. The microinjected embryos are allowed to hatch to thereby obtain G₀ transgenic chimeric birds, which are further grown and mated to give G₁. The efficiency of transgene transmission from G₀ to G₁ is considered to be dependent on the proportion of germ cells having the vector-derived gene arrangement among all germ cells that G₀ have. The efficiency of vector-derived gene transmission from G₀ to G₁ as so far reported is less than 10% and it is therefore necessary to test a large number of G₁ birds for presence or absence of the transgene, and this is a great obstacle to the production of transgenic birds. Further, the fact that the efficiency of transgene transmission from G₀ to G₁ as attained so far is as low as less than 10% indicates that the number of copies of the gene transmitted to and transduced into G₁ transgenic birds is small, and this constitutes a factor restricting the productivity in producing useful substances in transgenic birds. In fact, there is no case report about the production of G₁ having a plurality of transgenes using a replication-defective retrovirus vector. The present invention discloses a method for obtaining G₀ transgenic chimeric birds showing higher transgene transmission efficiency than in the prior art as well as G₁ transgenic birds having a plurality of transgene copies.

As reported by Bosselman et al. (Bosselman, R. A. et al. (1989), Science, 243, 533), it has been reported that when a Reticuloendotheliosis virus-derived, replication-defective retrovirus vector system was used, an autonomously replicating, infectious retrovirus was detected among G₀. The present invention discloses a method of producing safer G₀ transgenic chimeric birds, transgenic birds and offspring thereof, which are free of the risk of autonomously replicating retrovirus generation.

The transgenic bird production technology is very important as a method of breeding in birds. The present invention discloses a method of avian breeding using a replication-defective retrovirus vector.

When transgenic birds produced by using a retrovirus vector capable of infecting birds are infected with the same retrovirus or a related retrovirus, there arises the risk of the provirus transduced being rescued as infective virus particles. The present invention discloses a method of transducing a highly safe Moloney murine leukemia virus (MoMLV)-derived retrovirus vector, which is used in gene therapy in humans, into birds.

A retrovirus is an RNA virus and, through the process called infection, it gets into a host cell, and is converted into a double-stranded DNA by reverse transcriptase. The DNA is then integrated (inserted) into the genome of the host cell under the action of integrase derived from the viral pol. The integrated retrovirus is called provirus. The provirus is transferred to daughter cells as cell division proceeds. A retrovirus genomic RNA is transcribed from the provirus, and the retrovirus genomic RNA has the packaging signal sequence and it is taken up by a virus particle constituted of a group of proteins produced from the two genes, gag and pol, which the provirus has. The retrovirus genomic RNA-containing virus particle is enveloped by a host cell membrane containing a membrane protein transcribed and translated from the env gene which the provirus also has. The enveloped virus particle is released from the cell and the retrovirus is reproduced in an infectious form.

Retrovirus vectors that utilize such life cycle of retroviruses have been developed since the 1980's.

Retrovirus vectors are roughly classified into replication-competent retrovirus vectors and replication-defective retrovirus vectors.

Replication-competent retrovirus vectors contain the three functional genes gag, pol and env, which are necessary for the replication of virus particles. Such replication-competent retrovirus vectors involve the risk of their producing infectious virus particles in animal individuals harboring them as transduced therein to thereby infect other living organisms, hence they are not suited for use in producing industrially useful transgenic animals.

Replication-defective retrovirus vectors are lacking in at least one of the three functional genes (gag, pol and env) necessary for the replication of virus particles, or at least one of them does not function. Therefore, once a target cell has been infected with such a vector, the target cell does not generate any new infectious virus particle. Replication-defective retrovirus vectors developed in recent years are lacking in all of the genes gag, pol and env. Various methods are known for preparing such replication-defective retrovirus vectors. Basically, a vector construct and a system (e.g. helper virus or packaging cells) providing the gag, pol and env gene products necessary for the production of infectious virus particles are required.

The vector construct is a DNA having a structure such that the functional genes, such as gag, pol and env, have been removed from the provirus structure and a desired gene or gene arrangement has been inserted instead of the functional genes. The vector construct has a packaging signal sequence ψ. The packaging cell is a cell producing an infectious virus particle upon transduction of the vector construct, and the functional gag, pol and env genes are expressed in that cell.

When the vector construct is transduced into packaging cells, the replication-defective retrovirus vector can be recovered from the broth of the cells.

A foreign gene can be efficiently transduced into target cells by the retrovirus vector through the processes of infection of the cells and integration into the genome. This process of infection depends on the envelope protein of the retrovirus vector and the envelope protein receptor existing in the membrane of the target cells. Therefore, when the target cells are completely lacking or scanty in retrovirus vector envelope protein receptor, the gene transduction using the retrovirus vector cannot be made or, if it is possible, the efficiency is poor.

For example, MoMLV, a typical retrovirus, includes the ecotropic virus and amphotropic virus, which differ in envelope protein. The former infects mouse and rat cells alone but does not infect hamster-derived BHK cells. The latter infects not only mouse and rat cells but also hamster, human and simian cells, among others.

MoMLV-derived retrovirus vectors have been studied since the 1980's and have made it possible to transduce a gene stably into mammalian cells.

MoMLV-derived retrovirus vectors are highly safe vectors used in human gene therapy. However, retrovirus vectors, typically MoMLV-derived retrovirus vectors, are characterized in that the efficiency of infection (efficiency of gene transduction) of target cells greatly varies depending on the target cell species and that the infection and gene transduction are very difficult in certain target cell species.

Another characteristic feature of retrovirus vectors of this type is that the virus envelope is weak, so that such a concentration procedure as ultracentrifugation fails to increase the virus titer in some instances. In other instances, ultracentrifugation or a like concentration procedure rather decreases the virus titer.

G₀ transgenic chimeric bird production includes a process of microinjection of a retrovirus vector into the avian embryo, and the liquid amount capable of being microinjected into the embryo depends on the size of the avian embryo. In reality, when the embryo is at the blastoderm stage, the limit is several microliters in quails, or ten and several microliters in chickens.

As discussed above, it was considered that the production of G₀ transgenic chimeric birds high in the efficiency of transgene transmission using a retrovirus vector might be influenced by the sensitivity or nonsensitivity of the primordial germ cells or precursor cells thereof contained in the avian embryo to the retrovirus vector, the titer of the retrovirus vector stock to be used and liquid amount to be microinjected into the embryo.

An important means for modifying a retrovirus vector with regard to the range of hosts thereof is the use of a retrovirus vector whose envelope protein, which determines the range of hosts thereof, has been replaced by the envelope protein derived from another virus (such a retrovirus vector is referred to as pseudotype retrovirus vector). Emi et al. (Emi, N, et al. (1991), Virology, 65, 1202) produced a pseudotype retrovirus vector having the VSV-G protein, which is the envelope protein of vesicular stomatitis virus (VSV), in lieu of the envelope protein of MOMLV and showed that it can infect and be transduced into BHK cells, which are intrinsically low in sensitivity to MoMLV infection. Later, Burns et al. (Burns, J. C. et al. (1993), Proc. Natl. Acad. Sci. USA, 90, 8033) made improvements and showed that the pseudotype retrovirus vector having the VSV-G protein can be concentrated by ultracentrifugation. Similarly, it is reported that the range of hosts of a pseudotype MOMLV having the Sendai virus-derived hemagglutinin-neuraminidase or a fused protein (SV-F) as the envelope protein is broadened or conversely narrowed, respectively (Spiegel, M. et al. (1998), J. Virol., 72, 5296).

It is known that VSV infects most of mammalian and avian cultured cells. It is also known that it infects and propagates in cultured cells of reptiles, fish, and insects such as mosquitoes and drosophilae, for instance.

The present inventors made investigations as to whether a pseudotype retrovirus vector (replication-defective virus) having the VSV-G envelope protein micro injected into the embryo of birds could infect and be transduced into the precursor cells of germ cells or not. As a result, it was found that the G₀ transgenic chimeric birds obtained can transmit the transgene to G₁ with unusually very high efficiency. This finding has now led to completion of the present invention.

The present inventors further found that G₀ according to the invention, which have a very high level of efficiency of transgene transmittance to G₁, are capable of transmitting a large number of copies of the transgene to G₁ transgenic birds. This finding is very important in increasing the production of a useful substance in transgenic birds.

The fact that G₀ transgenic chimeric birds very highly efficient in gene transmission to G₁ can be obtained in accordance with the present invention provides an excellent method for the production of transgenic birds into which a gene whose function is unknown is efficiently transduced in order to identify the function of that gene or the function of a protein encoded by that gene.

Further, G₀ transgenic chimeric birds produced according to the present invention using a pseudotype retrovirus vector having the VSV-G envelope protein do not release infectious particles at all, hence G₁ and other birds will not be contaminated with infectious virus particles. This method of producing transgenic birds can thus be said to be a safe one.

Furthermore, transgenic birds produced by using a retrovirus vector whose basic skeleton is MOMLV, which are disclosed for the first time by the present invention, are very low in the risk of the gene transduced by the retrovirus capable of infecting birds being rescued as infectious virus particles to thereby infect or be transmitted to other birds. Thus, the relevant method is a safer method of producing transgenic birds.

The present inventors found that G₀ transgenic chimeric birds produced by using a pseudotype retrovirus vector having the VSV-G envelope protein have a large number of transgene copies in the germ cell lineage genome. The sites of transgene insertion are apparently at random. The possibility was considered that when a site of transgene insertion is in an avian functional gene arrangement, G₁ transgenic birds modified in the function of the gene might be born efficiently from G₀ transgenic chimeric birds. For example, it was considered that G₁ transgenic birds modified in feather color tone might be born efficiently. The present inventors succeeded in obtaining G₁ transgenic birds showing an albino character by mating from G₀ transgenic chimeric birds produced by using a pseudotype retrovirus vector having the VSV-G envelope protein. This indicates that birds modified in gene function or birds having a knockout gene can be produced and bred efficiently. This finding has led to completion of the present invention in another aspect.

Thus present invention is thus consists in a G₀ transgenic chimeric bird
which comprises a G₀ transgenic chimeric bird resulting from gene transduction using a replication-defective retrovirus vector and that efficiency of transgene transmission to G₁ is not less than 10%.

### BRIEF DESCRIPTION OF THE DRAWINGS

FIG. 1 shows the structure of a replication-defective retrovirus vector-derived vector construct, pLGRN. Neo^{r} denotes the neomycin resistance gene. P_{RSV} denotes the Rous sarcoma virus promoter sequence. GFP denotes the green fluorescent protein gene. ψ+ indicates the existence of a packaging signal sequence. 5′LTR and 3′LTR denote the MOMLV long terminal repeat sequences, respectively.

FIG. 2 shows the results of testing for the existence of a transgene in G₀ transgenic chimeric quails by the PCR method. C indicates a positive control. Neo^{r} indicates the neomycin resistance gene.

FIG. 3 shows the results of testing, by the PCR method, for the vector transduced in various tissues of G₁ transgenic quails. M indicates a marker, C1 a positive control, and C2 a negative control. L, H, T, P, B and S indicate liver, heart, gonad, spleen, brain and epidermis, respectively. Neo^{r} denotes the neomycin resistance gene, and GFP denotes the green fluorescent protein gene.

FIG. 4 shows the results of transgene analysis in G₁ transgenic quails by Southern blotting. For lanes 1 to 15, Southern blotting was carried out using a GFP probe. For lanes 16 to 23, Southern blotting was carried out using a Neo^{r} probe. For lanes 1 to 7, XhoI-cleaved DNAs were used and, for lanes 8 to 23, KpnI-cleaved DNAs were used. Lanes 1 to 6, 9 to 14 and 17 to 22, show the results of Southern blotting using the respective probes following electrophoresis of DNAs from 6 G₁ transgenic quails after cleavage with the respective restriction enzymes, lanes 7, 15 and 23 show the results obtained in the same manner using DNAs from genetically unengineered quails (negative controls), and lanes 8 and 16 show the results in the same manner using the replication-defective retrovirus vector-derived vector construct pLGRN (positive control).

FIG. 5 shows the results of analysis, by the RT-PCR method, of transgene expression in various tissues in G₁ transgenic quails and G₂ transgenic quails. m indicates a marker. H, B, L, M, K, S and G indicate heart, brain, liver, muscle, kidney, spleen and gonad, respectively.

### DETAILED DISCLOSURE OF THE INVENTION

In the following, the present invention is described in detail.

The replication-defective retrovirus vector is not particularly restricted but may be any of those lacking in replicating ability. Thus, there maybe mentioned, for example, those which are lacking in at least one of the three functional genes (gag, pol and env) that are necessary for virus particle replication or those in which at least one of the genes does not function. Such retrovirus vectors lacking in at least one of gag, pol and env or lacking in the function of at least one of them, once they have infected target cells, can no more form new infectious virus particles.

The gene gag codes for the virus particle structural proteins, namely matrix, capsid and nucleocapsid, pol for the enzymes, namely reverse transcriptase, integrase and protease, and env for the envelope protein.

Preferred as the replication-defective retrovirus vector to be used in the practice of the invention are, for example, those derived from Moloney murine leukemia virus (MOMLV), Rous sarcoma virus (RSV), mouse mammary tumor virus (MMTV) and the like. Among them, MoMLV-derived ones are preferred.

The above-mentioned MoMLV is a virus that has served as the basis for the development of a large number of retrovirus vectors. It has, as its genome, a single-stranded RNA having a length of about 8 kilobases. Its structure is similar to the structure of a eukaryotic mRNA. Thus, it has a cap structure at the 5′ terminus, and a poly(A) tail at the 3′ terminus. At the 5′ terminus and 3′ terminus, there are regions necessary for replication and transcription, namely R-U5 at the 5′ end and U3-R at the 3′ end. Between these both termini, there are coding regions, gap, pol and env. Between U5 and gag, there is the packaging signal sequence ψ, which is necessary for the viral RNA genome being taken up into the virus particle. At the time of infection, MOMLV intrudes into cells. The viral genome that has intruded is converted to a double-stranded DNA by reverse transcriptase, and the DNA is inserted into the host cell genome. The thus-inserted virus-derived DNA is called provirus. The viral genomic RNA is again synthesized based on the provirus under the action of the host cell RNA polymerase. All proteins that are necessary for the production of infectious MoMLV particles are produced based on gag, pol and env, and MoMLV is released from the cells upon germination.

Generally, various methods are known for the preparation of replication-defective retrovirus vectors (Retroviruses, Coffin, J. M., Hughes, S. H. and Vermus, H. E. eds. (1997), Cold Spring Harbor Laboratory Press). Basically, a vector construct and a system (e.g. helper virus or packaging cells) providing the gag, pol and env gene products necessary for the production of infectious virus particles are required.

Generally used as the above-mentioned system (e.g. helper virus or packaging cells) for providing the gag, pol and env gene products are those systems which are described in Retroviruses, Coffin, J. M., Hughes, S. H. and Vermus, H. E. eds. ((1997), Cold Spring Harbor Laboratory Press), or the like. Most often used among others are cells (packaging cells) constitutively producing the gag, pol and env gene products. When the gene arrangement involving gag, pol and env exists in the packaging cells in a manner structurally similar to the retrovirus, there arises the possibility that the sequences may undergo recombination with the vector construct inserted into the packaging cells, possibly leading to the formation of a replication-competent retrovirus. Therefore, the packaging cells used in recent years are cells resulting from transformation with two expression vectors, namely gag-pol and env, and allowing constitutive or transient expression of the gag-pol and env.

The method of transducing the vector construct into packaging cells is not particularly restricted but includes, among others, the lipofection method, calcium phosphate method, and electroporation method.

Suited for use as the replication-defective retrovirus vector in the practice of the present invention are replication-defective retrovirus vectors having a VSV-G protein-containing membrane. By substituting the VSV-G protein for the envelope protein of a replication-defective retrovirus vector, it becomes possible for birds to be used as hosts of the vector even when it is derived from a virus incapable of infecting birds.

The method of preparing the defective retrovirus vector having a VSV-G protein-containing membrane is not particularly restricted but may comprise, for example, using packaging cells expressing the VSV-G protein in lieu of the env-expressing packaging cells, transducing a vector construct into such packaging cells and culturing the packaging cells. The desired vector can be recovered from the culture broth.

Suited for use as the VSV-G protein-expressing packaging cells are cells obtained by transfection of packaging cells constitutively expressing gag-pol with a VSV-G protein expression vector. On the occasion of transfection of packaging cells constitutively expressing gag-pol with a VSV-G protein expression vector, cotransfection with the vector construct may be carried out simultaneously (Yee, J. K. et al. (1994), Methods Cell Biol., 43, Pt A, 99). It is also possible to use packaging cells constitutively expressing gag-pol and capable of inducible expression of a large amount of the VSV-G protein under certain conditions (Arai, T. et al. (1998), J. Virol., 72, 1115; U.S. Pat. No. 5,739,018).

The replication-defective retrovirus vector having a VSV-G protein-containing membrane may also be prepared by transfecting packaging cells having a replication-defective provirus and constitutively expressing gag-pol with a VSV-G protein expression vector, or may be prepared in a cell-free system (Abe, A. et al. (1998), J. Viol., 72, 6356).

Since the VSV-G protein is toxic to cells, cells constitutively expressing a large amount of the VSV-G protein in a stable manner cannot be obtained. Therefore, a vector construct containing the VSV-G gene is transduced into packaging cells constitutively expressing the gag-pol gene, whereby a retrovirus having the VSV-G protein and envelope protein is recovered (Emi, N. et al. (1991), Virology, 65, 1202; Burns, J. C. et al. (1993), Proc. Natl. Acad. Sci. USA, 90, 8033). However, the pseudotype retrovirus vector produced in that manner contains the VSV-G gene, which is unnecessary, so that it is not suited for use in producing transgenic birds.

The transgene to be transduced into birds in accordance with the present invention is not particularly restricted but preferably is a non-retrovirus-derived gene.

The above non-retrovirus-derived gene is not particularly restricted but includes, among others, the neomycin resistance gene and the green fluorescent protein (GFP) gene. Genes coding for useful proteins and so forth may also be used.

The transgene is inserted at a site between the 5′ terminus and 3′ terminus of the provirus in the vector construct. For expression of the transgene in transgenic birds, a promoter sequence, which controls the gene transcription, may be used according to need.

Utilizable as the promoter sequence are promoter sequences controlling tissue-specific expression, promoter sequences controlling constitutive expression in tissues, or inducible promoters.

The G₀ transgenic chimeric bird according to the invention is not particularly restricted but includes, among others, useful birds raised as farm animals, for example chickens, ducks, turkeys, wild ducks, ostriches and quails. Among them, chickens and quails are preferred. Chickens and quails are readily available.

The method of producing the G₀ transgenic chimeric bird according to the invention is not particularly restricted but may comprise, for example, transducing a defective retrovirus vector having a VSV-G protein-containing membrane into an avian embryo and hatching the same.

The method of transducing the replication-defective retrovirus vector having a VSV-G protein-containing membrane into an avian embryo is not particularly restricted but mention may be made of the method comprising microinjecting the replication-defective retrovirus vector into the embryo after oviposition.

Applicable as the method of microinjection are the methods conventional in the art. Thus, applicable are the methods disclosed in the literature by Bosselman et al. (Bosselman, R. A. et al. (1989), Science, 243, 533) and Vick et al. (Vick, L. et al. (1993), Proc. R. Soc. Lond. B Biol. Sci., 251, 179), respectively, as well as the method shown herein in the example section by the present inventors.

The above-mentioned method of producing G₀ transgenic chimeric birds also constitutes one aspect of the present invention.

For incubating the embryo microinjected with the replication-defective retrovirus vector and hatching a G₀ transgenic chimeric bird, the method using an artificial eggshell as developed by the present inventors (Kamihira, M. et al. (1998), Develop. Growth Differ., 40, 449) and the methods disclosed in the literature by Bosselman et al. (Bosselman, R. A. et al. (1989), Science, 243, 533) or Vick et al. (Vick, L. et al. (1993), Proc. R. Soc. Lond. B Biol. Sci., 251, 179) can be applied.

By growing the G₀ transgenic chimeric bird according to the invention to an imago and mating the same with non-transgenic birds, it is possible to transmit the gene transduced into the G₀ transgenic chimeric bird to G₁ birds. The success or failure in gene transmission can be judged by extracting DNA from the blood or each tissue, for instance, of the G₁ obtained and checking the DNA for the presence or absence of the transgene by the PCR method or hybridization method, for instance.

The G₀ transgenic chimeric bird according to the invention comprises the efficiency of transgene transmission to G₁ is not lower than 10%. The efficiency of gene transmission is expressed in terms of the proportion (%) of those G₁ transgenic birds which have the transgene to all G₁ birds obtained by mating of the G₀ transgenic chimeric bird. Preferably, the efficiency is 20 to 90%.

The method of producing transgenic birds which comprises transducing a MoMLV-derived, replication-defective retrovirus vector into an avian embryo, hatching the embryo, further growing the thus-obtained G₀ transgenic chimeric bird and subjecting the same to mating, and the thus-obtained transgenic birds, as well as the method of producing transgenic birds which comprises transducing a replication-defective retrovirus vector having a VSV-G protein-containing membrane into an avian embryo, hatching the embryo, further growing the thus-obtained G₀ transgenic chimeric bird and subjecting the same to mating, and the thus-obtained transgenic birds also constitute respective aspects of the present invention.

In the present specification, the transgenic birds include, within the meaning thereof, offspring thereof.

The transgenic bird of the present invention has the transgene in all germ cells and somatic cells, and the transgene that said transgenic bird has is transmitted to its offspring obtained by mating thereof.

Preferably, the transgenic bird of the invention has a plurality of copies of the transgene.

The number of copies of the transgene which the transgenic bird of the invention has can be confirmed by the quantitative PCR method or by cleaving DNA of that bird with an appropriate restriction enzyme, followed by Southern blotting. The number of transgene copies which the transgenic bird of the invention has is preferably not less than 2.

The transcription and expression of the transgene in the transgenic bird of the invention can be confirmed by extracting mRNA from each tissue of the transgenic bird and carrying out the RT-PCR method, or can be confirmed also by the antigen-antibody reaction, for instance.

The inherited character of the offspring obtained from the G₀ transgenic chimeric bird by mating can be confirmed by checking for the character in question (e.g. feather color tone, growth rate, feed efficiency, sex ratio, meat quality, fecundity and longevity in or among the offspring).

If necessary, the transgenic bird of the invention may differ in inherited character from the parent birds. The inherited character differing from the counterpart in the parent birds is not particularly restricted but may be “albino”, for instance. Since albino is a character resulting from disruption of the tyrosinase gene on the Z chromosome, the development of albino individuals indicates that the rate of transmission of the transgene is high.

According to the present invention, it is possible to breed birds having a desired character. The present invention can thus be utilized in efficiently producing and breeding birds modified in gene function or birds having a knockout gene. The invention can also be used in producing useful substances.

### BEST MODES FOR CARRYING OUT THE INVENTION

The following examples illustrate the present invention in further detail. These examples are, however, by no means limitative of the scope of the present invention. The sensitivity of the primordial germ cells or precursors thereof as contained in the embryo of birds used in the examples to the replication-defective retrovirus vectors, the titers of the viral solutions, the liquid amounts used in microinjection into embryos and other details in the examples are by no means limitative of the scope of the invention.

### EXAMPLE 1

### Preparation of a Replication-Defective Retrovirus Vector

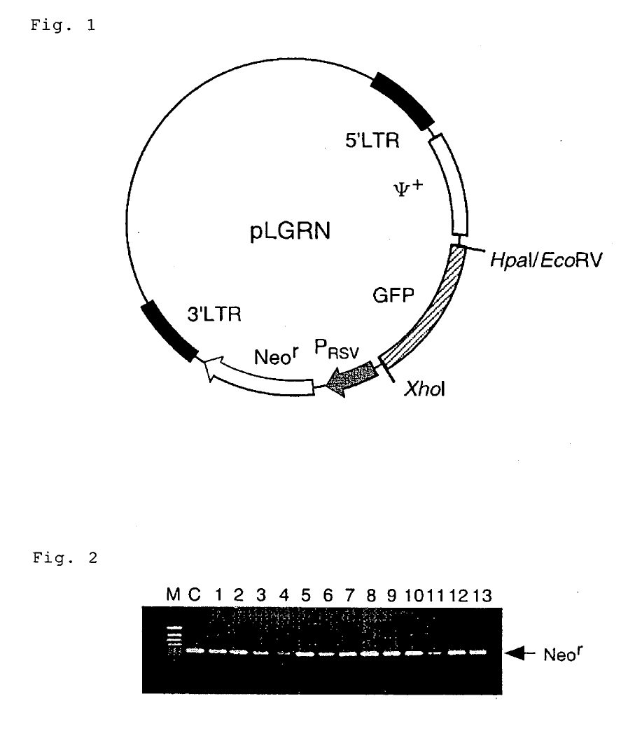

A replication-defective retrovirus vector-derived vector construct named pLGRN was produced in the following manner. Thus, the green fluorescent protein (GFP) gene was excised from the plasmid pGREEN LANTERN (product of Gibco BRL) with the restriction enzyme NotI and inserted into the NotI site of pZeoSV2 (+) (product of Invitrogen), whereby a plasmid named pZeo-GFP was produced. Then, the GFP gene was further excised from pZeo-GFP using the restriction enzymes EcORV and XhoI and insertedinto the HpaI-XhoI site of pLXRN (product of Clontech), whereby the vector construct pLGRN was produced. The structure of the thus-produced replication-defective retrovirus vector-derived vector construct pLGRN is shown in FIG. 1.

### EXAMPLE 2

### Production of a Replication-Defective Retrovirus Vector by Cotransfection

On the day before transfection, a dish, 100 mm in diameter, was inoculated with 5×10⁶ GP293 cells (product of Clontech), which are virus packaging cells. After 24 hours of incubation, it was confirmed that GP293 cells had grown to about 80% confluency. The medium was then replaced with a fresh portion of DMEM (Dulbecco's modified Eagle's medium). The VSV-G expression vector pVSV-G (product of Clontech; 8 μg) and 8 μg of pLGRN were transduced into GP293 cells by the lipofection method. After 48 hours, the virus particle-containing culture supernatant was recovered and removed contaminants by passing it through a 0.45 μm cellulose acetate filter. Polybrene was added to the solution obtained, which contained the virus having the VSV-G envelope protein, to a concentration of 10 μg/ml. The thus-prepared viral solution had a titer of about 10⁵ cfu (colony forming units). The viral titer measurement was carried out as shown below by way of example. On the day before assaying, four dishes, 35 mm in diameter, were each inoculated with 7×10⁴ NIH3T3 cells (American Type Culture Collection), followed by incubation. 10²-fold to 10⁶-fold dilutions of the viral solution were added to the dishes in an amount of 1 ml per dish and, two days later, the proportion of cells expressing GFP in each dish was determined under a fluorescence microscope, and the titer was calculated as follows:
Example: number of cells (10⁵)×dilution factor (10⁴)×expression rate (0.8)=8×10⁸ cfu/ml.

### EXAMPLE 3

### Establishment of a Stable Transformant Strain for Replication-Defective Retrovirus Vector Production

GP293 cells were prepared as in Example 2. The culture medium was removed from the dish in which GP293 cells had grown, and 10 ml of the VSV-G envelope protein-containing virus solution prepared in Example 2 was added. After 2 days of further incubation, the GP293 cells treated for viral infection was subcultured in a culture medium containing 600 μg/ml of G418. A stable, G418-resistant transformant strain was thus obtained.

### EXAMPLE 4

### Preparation of a High-Titer, Replication-Defective Retrovirus Vector

The stable, G418-resistant transformant strain obtained in Example 3 was cultured in a dish, 100 mm in diameter until an about 80% confluency, and 16 μg of pVSV-G was transduced thereinto by the lipofection method. After 48 hours, the virus particle-containing culture supernatant was recovered. The titer of the virus contained in this culture supernatant was about 10⁷ cfu/ml.

### EXAMPLE 5

### Concentration of the Replication-Defective Retrovirus Vector

The replication-defective retrovirus vector-containing culture supernatant obtained in Example 4 was centrifuged at 50,000×g at 4° C. for 1.5 hours to form sediment. The supernatant was removed, 50 μl of 50 mM Tris-HCl (pH 7.8) was added to the virus particle-containing sediment, and a 130 mM NaCl plus 1 mM EDTA solution was added. After overnight standing of the mixture at 4° C., thorough suspension was caused and the viral solution was recovered. The thus-prepared virus had a titer of about 10⁹ cfu/ml.

### EXAMPLE 6

### Microinjection of the Viral Solution into Quail Embryos

Fertile WE strain quail eggs (obtained from Nippon Institute for Biological Science) were used. The eggshell of each fertile egg was sterilized with 70% ethanol and a circle with a diameter of 2 cm was cut off from the sharp end portion using a diamond cutter (MINOMO 7C710, product of Minitor) to expose the embryo. While observing the blastoderm under a stereomicroscope, the blastoderm was stuck with a needle worked from a glass tube (CD-1, product of Olympus) on a micropipet manufacturing machine (PC-10, product of Olympus) by breaking the tip to attain an outside diameter of about 20 μm, and about 2 μl of the viral solution prepared in Example 5 was microinjected into the middle of the subgerminal cavity using a microinjector (Transjector 5246, product of Eppendorf).

### EXAMPLE 7

### Quail Embryo Culture

The fertile quail eggs microinjected with the virus particles in Example 6 were each filled with egg white to the cut end of the eggshell and then covered with a Teflon membrane (Milliwrap, product of Millipore) and a polyvinylidene chloride wrap (Saran Wrap, Product of Asahi Kasei) using egg white as a paste. They were incubated in an incubator (model P-008, product of Showa Furanki Kenkyusho) with a built-in automatic egg rotator at 37.9° C. and 65% humidity for about 48 hours while rotating the eggs by 90 degrees at 15-minute intervals. After confirmation of the normal progress of development, each virus-infected embryo was transferred to an S-size hen's eggshell through a hole, 4 cm in diameter, made at the sharp end portion of the eggshell. The embryo was placed on top and allowed to be in contact with air, 0.5 ml of a 50 mg/ml calcium lactate suspension in egg white was added, and the eggshell was then tightly closed with a wrap using egg white as a paste. The embryos treated in this way were again placed in the incubator and incubated at 37.9° C. and 65% humidity for 13 days while rotating the eggs by 30 degrees at 1-hour intervals. Then, the rotating was stopped and the eggs were allowed to stand. When an embryo began pulmonary respiration, a tiny hole was made in the wrap using a needle to thereby assist the respiration. After disappearance of blood from the chorioallantoic membrane, the nestling was taken out of the incubator to complete hatching.

### EXAMPLE 8

### Hatchability of Transgenic Quail Embryos

The virus-infected embryo incubation procedure was repeated three times (40 to 49 embryos per experiment), and the embryos treated for gene transduction with the replication-defective retrovirus vector were allowed to hatch by the method shown in Example 7. In the three experiments, the transgenic quail embryos could be hatched with a hatchability of 13 to 39%. The hatchability data for the transgenic quail embryos are shown in Table 1.

TABLE 1

| | Number of | Survival rate | |
|---|---|---|---|
| Experiment No. | embryos treated | on day 3 | Hatchability |
| 1 | 40 | 36 (90%) | 12 (30%) |
| 2 | 49 | 44 (90%) | 19 (39%) |
| 3 | 45 | 29 (64%) | 6 (13%) |

### EXAMPLE 9

### Testing of the Transgene in the Quails that Had Hatched

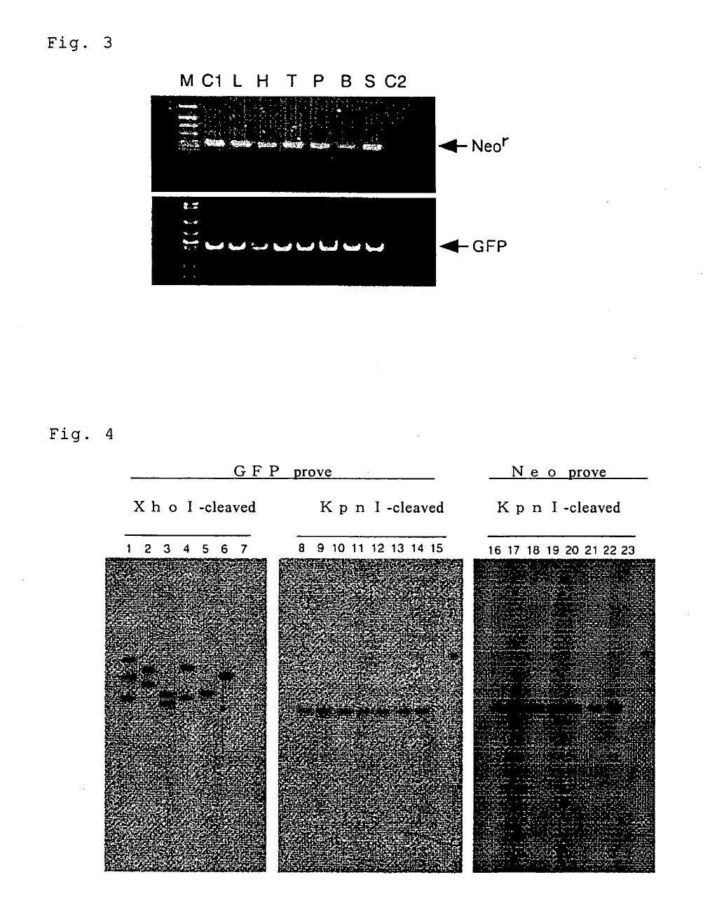

The chorioallantoic membrane was collected from each quail that had hatched in Example 8, and genomic DNA was extracted therefrom using Mag Extractor -genome- (product of Toyobo). The presence or absence of the transgene was tested by amplifying, by the PCR method, apart (368 bp) of the neomycin resistance gene contained in the replication-defective retrovirus vector used for gene transduction. Amplification of the neomycin resistance gene could be confirmed for the chorioallantoic membranes of all 13 quails subjected to testing (FIG. 2). This indicates that all the 13 quails tested were G₀ transgenic chimeric quails.

### EXAMPLE 10

### Efficiency of Transgene Transmission to Offspring of the G₀ Transgenic Chimeric Quails

Six of the G₀ transgenic chimeric quails that had hatched in Example 8 were mated with genetically unengineered quails, whereby a plurality of G₁ quails were obtained. Genomic DNA was prepared from the chorioallantoic membrane of each G₁ quail that had hatched and the gene transmission was confirmed by the PCR method in the same manner as in Example 9. As shown in Table 2, G₁ transgenic quails were obtained with an average efficiency of 82%. The efficiency of transgene transmission to G₁ as shown in a G₀ (#6) group was 88%.

TABLE 2

| | | Number | | |
|---|---|---|---|---|
| G₀ | | of G1 | Number of | |
| # | Sex | tested | transmission cases | Efficiency (%) |
| 1 | ♀ | 23 | 20 | 87 |
| 2 | ♀ | 18 | 15 | 83 |
| 3 | ♀ | 21 | 16 | 76 |
| 4 | ♂ | 16 | 13 | 81 |
| 5 | ♂ | 20 | 15 | 75 |
| 6 | ♀ | 17 | 15 | 88 |

| | | | |
|---|---|---|---|
| Total (average) | 115 | 94 | 82 |

### EXAMPLE 11

### Existence of the Transgene in Various Tissues of the G₁ Transgenic Quails

For those G₁ transgenic quails for which the existence of the transgene in the chorioallantoic membrane could be confirmed, genomic DNA was extracted from each of various tissues (liver, heart, gonad, spleen, brain, epidermis), and whether the transgene was present in the whole body or not was examined by the PCR method. The neomycin resistance gene and GFP gene existing on the replication-defective retrovirus vector transduced were both amplified from the DNA of each organ. Thus, it could be confirmed that the replication-defective retrovirus vector transduced was present in the cells of the whole body (FIG. 3).

### EXAMPLE 12

### Determination of the Number of Copies of the Transgene

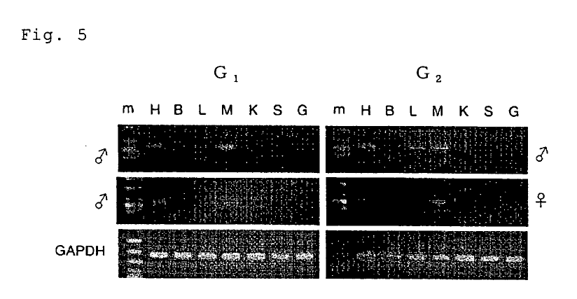

Genomic DNA was extracted from the blood of each of six G₁ transgenic quails. Each genomic DNA was cleaved with the restriction enzymes XhoI and KpnI, and the cleavage mixture was electrophoresed on a 0.8% agarose gel. After electrophoresis, the DNAs were alkali-transferred to a nylon membrane (Hydond N+, product of Amersham-Pharmacia). Southern hybridization was carried out using a GFP gene probe and neomycin resistance gene probe labeled with a radioisotope by the random primer method. The number of gene copies could be determined by cleavage with XhoI, and it was confirmed by cleavage with KpnI that the transgene had not undergone any deletion or recombination. The results of analysis of 6 G₁ transgenic quails are shown in FIG. 4. One individual had 3 copies of the transgene per genome, three had 2 copies, and one had one copy.

### EXAMPLE 13

### Transgene Expression in G₁ Transgenic Quails and G₂ Transgenic Quails

G₂ transgenic quails were obtained by mating the G₁ transgenic quails with genetically unengineered quails. mRNA was extracted from each of tissues (heart, brain, liver, muscle, kidney, spleen and gonad) of the G₁ transgenic quails and G₂ transgenic quails, and the mRNA was purified using an mRNA isolation kit (product of Roche). The expression of the neomycin resistance gene (amplified range 368 bp) and the expression of the GFP gene (amplified range 311 bp) were examined by the RT-PCR method (Ready to G₀ TR-PCR beads, product of Amersham-Pharmacia). The RT-PCR of the GAPDH gene (glyceraldehyde-3-phosphate dehydrogenase gene; amplified range 589 bp) was also carried out as a control. Relatively strong expression of the neomycin resistance gene was confirmed in heart and muscle. A certain extent of expression of that gene was also confirmed in liver and kidney. The expression of GFP was not detected by RT-PCR. In the G₂ transgenic quails as well, strong expression of the neomycin resistance gene was observed in heart and muscle, and the expression pattern had thus been transmitted from the G₁ transgenic quails to the G₂ transgenic quails. The results are shown in FIG. 5.

The fact that the expression of GFP was not observed means that the promoter activity of the LTR (long terminal repeat) of MOMLV does not function in birds. This suggests that the replication-defective retrovirus vector used in the practice of the invention is very safe in producing transgenic birds.

### EXAMPLE 14

### Production of Transgenic Birds Having the Character of Albino

Out of 16 G₁ transgenic quails derived from the G₀ transgenic chimeric quail (#4) as shown in Example 10, two were albino. Albino is the character resulting from disruption of the tyrosinase gene on the Z chromosome. It was thus suggested that the gene in question had been disrupted or the function thereof had been deleted by the above-mentioned replication-defective retrovirus vector.

### EXAMPLE 15

### Preparation of a Replication-Defective Retrovirus Vector to be Transduced into Chickens

The G418-resistant stable transformant strain obtained in Example 3 was cultured until about 80% confluency in a dish with a diameter of 100 mm, and 16 μg of pVSV-G was transduced thereinto by the lipofection method. After 48 hours, 12 ml of a virus particle-containing culture supernatant was recovered. This culture supernatant was centrifuged at 50,000×g at 4° C. for 1.5 hours to give sediment. The supernatant was removed, and 50 μl of a solution containing 50 mM Tris-HCl (pH 7.8), 130 mM NaCl and 1 mM EDTA was added to the virus particle-containing sediment. After overnight standing of the mixture at 4° C., thorough suspension was caused and the viral solution was recovered. The thus-prepared virus solution had a titer of about 1 to 2×10⁸ cfu/ml.

### EXAMPLE 16

### Microinjection of the Viral Solution into Chicken Embryos

Fertilized chicken eggs (obtained from Nippon Institute for Biological Science) were used. The eggshell of each fertilized egg was sterilized with 70% ethanol and a circle with a diameter of 3.5 cm was cut off from the sharp end portion using a diamond cutter (MINOMO 7C710, product of Minitor) to expose the embryo. While observing the blastoderm under a stereomicroscope, the blastoderm was stuck with a needle worked from a glass tube (CD-1, product of Olympus) on a micropipet manufacturing machine (PC-10, product of Olympus) by breaking the tip to attain an outside diameter of about 20 μm, and about 2 μl of the viral solution prepared in Example 15 was microinjected into the middle of the subgerminal cavity using a microinjector (Transjector 5246, product of Eppendorf).

### EXAMPLE 17

### Chicken Embryo Culture

The fertile chicken eggs microinjected with the virus particles in Example 16 were each filled with egg white to the cut end of the eggshell and then covered with a Teflon membrane (Milliwrap, product of Millipore) and a polyvinylidene chloride wrap (Saran Wrap, Product of Asahi Kasei) using egg white as a paste. They were incubated in an incubator (model P-008, product of Showa Furanki Kenkyusho) with a built-in automatic egg rotator at 37.9° C. and 65% humidity for about 48 hours while rotating the eggs by 90 degrees at 15-minute intervals. After confirmation of the normal progress of development, each virus-infected embryo was transferred to a double-yolked egg greater than the fertilized egg through a hole, 4.5 cm in diameter, made at the sharp end portion thereof. The embryo was placed on top and allowed to be in contact with air, 0.5 ml of a 50 mg/ml calcium lactate suspension in egg white was added, and the eggshell was then tightly closed with a wrap using egg white as a paste. The embryos treated in this way were again placed in the incubator and incubated at 37.9° C. and 65% humidity for 15 days while rotating the eggs by 30 degrees at 1-hour intervals. Then, the rotating was stopped and the eggs were allowed to stand. When an embryo began pulmonary respiration, a tiny hole was made in the wrap using a needle to thereby assist the respiration. After disappearance of blood from the chorioallantoic membrane, the chick was taken out of the incubator to complete hatching.

### EXAMPLE 18

### Hatchability of Transgenic Chicken Embryos

The virus-infected embryo incubation procedure was performed and the embryos treated for gene transduction with the replication-defective retrovirus vector were allowed to hatch by the method shown in Example 17. In this experiment, six chicks could be hatched (hatchability 17%) by incubation of 35 embryos.

### EXAMPLE 19

### Testing of the Transgene in the Chickens that Had Hatched

The chorioallantoic membrane was collected from each of six chickens that had hatched in Example 18, and genomic DNA was extracted therefrom using Mag Extractor -genome- (product of Toyobo). The presence or absence of the transgene was tested by amplifying, by the PCR method, apart (368 bp) of the neomycin resistance gene contained in the replication-defective retrovirus vector used for gene transduction. Amplification of the neomycin resistance gene could be confirmed for the chorioallantoic membranes of 4 chickens (67%) out of the six subjected to testing. This indicates that those chickens were G₀ transgenic chimeric chickens.

### EXAMPLE 20

### Efficiency of Transgene Transmission to Offspring of the G₀ Transgenic Chimeric Chickens

The four G₀ transgenic chimeric chickens (two males and two females) that had hatched in Example 19 were mated with genetically unengineered chickens, whereby the two female G₀ transgenic chimeric chickens gave a total of 19 G₁ chickens (4 and 15). Genomic DNA was prepared from the chorioallantoic membrane of each of the 19 G₁ chickens that had hatched and the DNA was amplified by the PCR method for checking the presence or absence of the transgene in the same manner as in Example 19. As a result, amplification of the neomycin resistance gene could be confirmed in one chicken (25%) and seven chickens (47%) obtained from the two G, transgenic chimeric chickens, respectively, and it was confirmed that these were G₁ transgenic chimeric chickens.

### INDUSTRIAL APPLICABILITY

The invention makes it possible to transduce a desired gene with very high efficiency and thus produce transgenic birds capable of expressing that gene. In particular, transgenic birds including such useful birds under breeding as chickens, ducks, turkeys, wild ducks, ostriches and quails can be produced with very high efficiency. According to the invention, safe transgenic birds, which will not release infectious virus particles, can be produced. Further, the invention provides a method of breeding birds having a desired character. Furthermore, according to the invention, it is possible to efficiently produce and breed birds modified in gene function or birds having a knockout gene and, thus, a method of producing transgenic birds for transducing a gene whose function is unknown into them and identify the function of the gene or the function of a protein encoded by the gene. According to the invention, it also becomes possible to produce useful substances efficiently.

1. A G₀ transgenic chimeric bird
which comprises a G₀ transgenic chimeric bird resulting from gene transduction using a replication-defective retrovirus vector and that efficiency of transgene transmission to G₁ is not less than 10%.

2. The G₀ transgenic chimeric bird according to claim 1,
wherein the replication-defective retrovirus vector is a vector derived from Moloney murine leukemia virus.

3. The G₀ transgenic chimeric bird according to claim 1 or 2,
wherein the transgene has a gene arrangement not derived from retrovirus.

4. The G₀ transgenic chimeric bird according to claim 3,
wherein the gene arrangement not derived from retrovirus is the neomycin resistance gene arrangement or green fluorescent protein gene arrangement.

5. The G₀ transgenic chimeric bird according to any of claims 1 to 4,
which is a chicken or quail.

6. A method of producing G₀ transgenic chimeric birds
which comprises efficiency of transgene transmission to G₀ is not less than 10% and
which comprises transducing a replication-defective retrovirus vector having a VSV-G protein-containing membrane into an avian embryo and hatching the embryo.

7. The method of producing G₀ transgenic chimeric birds according to claim 6,
wherein the replication-defective retrovirus vector is a vector derived from Moloney murine leukemia virus.

8. The method of producing G₀ transgenic chimeric birds according to claim 6 or 7,
wherein the transgene has a gene arrangement not derived from retrovirus.

9. The method of producing G₀ transgenic chimeric birds according to claim 8,
wherein the gene arrangement not derived from retrovirus is the neomycin resistance gene arrangement or green fluorescent protein gene arrangement.

10. The method of producing G₀ transgenic chimeric birds according to any of claims 6 to 9,
wherein the bird is a chicken or quail.

11. A method of producing transgenic birds
which comprises transducing a Moloney murine leukemia virus-derived, replication-defective retrovirus vector into an avian embryo, hatching the embryo, further growing the transgene-containing G₀ transgenic chimeric bird obtained and causing the same to mate.

12. The method of producing transgenic birds according to claim 11,
wherein efficiency of transgene transmission from the G₀ transgenic chimeric bird to G₁ is not less than 10%.

13. The method of producing transgenic birds according to claim 11 or 12,
wherein the transgenic bird has a plurality of copies of the transgene.

14. The method of producing transgenic birds according to any of claims 11 to 13,
wherein the transgene has a gene arrangement not derived from retrovirus.

15. The method of producing transgenic birds according to claim 14,
wherein the gene arrangement not derived from retrovirus is the neomycin resistance gene arrangement or green fluorescent protein gene arrangement.

16. The method of producing transgenic birds according to any of claims 11 to 15,
wherein the transgenic bird has an inherited character differing from the parent birds.

17. The method of producing transgenic birds according to claim 16,
wherein the inherited character differing from the parent birds is albino.

18. The method of producing transgenic birds according to any of claims 11 to 17,
wherein the bird is a chicken or quail.

19. A transgenic bird obtained by transducing a Moloney murine leukemia virus-derived, replication-defective retrovirus vector into an avian embryo, hatching the embryo, further growing the transgene-containing G₀ transgenic chimeric bird obtained and causing the same to mate.

20. The transgenic bird according to claim 19,
wherein the efficiency of transgene transmission from the G₀ transgenic chimeric bird to G₁ is not less than 10%.

21. The transgenic bird according to claim 19 or 20,
which has a plurality of copies of the transgene.

22. The transgenic bird according to any of claims 19 to 21,
wherein the transgene has a gene arrangement not derived from retrovirus.

23. The transgenic bird according to claim 22,
wherein the gene arrangement not derived from retrovirus is the neomycin resistance gene arrangement or green fluorescent protein gene arrangement.

24. The transgenic bird according to any of claims 19 to 23,
which has an inherited character differing from the parent birds.

25. The transgenic bird according to claim 24,
wherein the inherited character differing from the parent birds is albino.

26. The transgenic bird according to any of claims 19 to 25,
which is a chicken or quail.

27. A method of producing transgenic birds
which comprises transducing a replication-defective retrovirus vector having a VSV-G protein-containing membrane into an avian embryo, hatching the embryo, further growing the transgene-containing G₀ transgenic chimeric bird obtained and causing the same to mate.

28. The method of producing transgenic birds according to claim 27,
wherein the efficiency of transgene transmission from the G₀ transgenic chimeric bird to G₁ is not less than 10%.

29. The method of producing transgenic birds according to claim 27 or 28,
wherein the transgenic bird has a plurality of copies of the transgene.

30. The method of producing transgenic birds according to any of claims 27 to 29,
wherein the replication-defective retrovirus vector is a vector derived from Moloney murine leukemia virus.

31. The method of producing transgenic birds according to any of claims 27 to 30,
wherein the transgene has a gene arrangement not derived from retrovirus.

32. The method of producing transgenic birds according to claim 31,
wherein the gene arrangement not derived from retrovirus is the neomycin resistance gene arrangement or green fluorescent protein gene arrangement.

33. The method of producing transgenic birds according to any of claims 27 to 32,
wherein the transgenic bird has an inherited character differing from the parent birds.

34. The method of producing transgenic birds according to claim 33,
wherein the inherited character differing from the parent birds is albino.

35. The method of producing transgenic birds according to any of claims 27 to 34,
wherein the bird is a chicken or quail.

36. A transgenic bird obtained by transducing a replication-defective retrovirus vector having a VSV-G protein-containing membrane into an avian embryo, hatching the embryo, further growing the transgene-containing G₀ transgenic chimeric bird obtained and causing the same to mate.

37. The transgenic bird according to claim 36,
wherein the efficiency of transgene transmission from the G₀ transgenic chimeric bird to G₁ is not less than 10%.

38. The transgenic bird according to claim 36 or 37,
which has a plurality of copies of the transgene.

39. The transgenic bird according to any of claims 36 to 38,
wherein the replication-defective retrovirus vector is a vector derived from Moloney murine leukemia virus.

40. The transgenic bird according to any of claims 36 to 39,
wherein the transgene has a gene arrangement not derived from retrovirus.

41. The transgenic bird according to claim 40,
wherein gene arrangement not derived from retrovirus is the neomycin resistance gene arrangement or green fluorescent protein gene arrangement.

42. The transgenic bird according to any of claims 36 to 41,
which has an inherited character differing from the parent birds.

43. The transgenic bird according to claim 42,
wherein the inherited character differing from the parent birds is albino.

44. The transgenic bird according to any of claims 36 to 43,
which is a chicken or quail.
